(19) United States
(12) Patent Application Publication
(10) Pub. No.: US 2026/0355543 A1
(43) Pub. Date: Nov. 12, 2026

(54) SMART ADAPTIVE NEUROMODULATION DEVICE FOR STELLATE GANGLION MODULATION

(71) Applicant: Hersh Patel, Wilmington, DE (US)

(72) Inventor: Hersh Patel, Wilmington, DE (US)

(21) Appl. No.: 19/736,446

(22) Filed: Jul. 9, 2026

Related U.S. Application Data
(62) Division of application No. 19/092,012, filed on Mar. 27, 2025.

(51) Int. Cl. A61N 7/00; A61B 5/00; A61B 5/024; A61B 5/0531; G16H 20/40; G16H 40/67

(52) U.S. Cl. CPC A61N 7/00; A61B 5/02405; A61B 5/0531; A61B 5/4836; G16H 20/40; G16H 40/67; A61N 2007/0026

(57) ABSTRACT

A non-invasive neuromodulation device for modulating a stellate ganglion may include a wearable collar configured to be positioned around a user's neck; a transducer array integrated into the collar, wherein the transducer array includes at least one low-intensity focused ultrasound (LIFU) emitter; one or more biometric sensors configured to measure physiological parameters including heart rate variability and skin conductance; a control unit with a microprocessor configured to receive real-time biometric data from the sensors; and a feedback system operatively connected to the control unit, wherein the control unit dynamically adjusts neuromodulation parameters of the transducer array based on received biometric data to optimize sympathetic nervous system regulation.

### CROSS-REFERENCE TO RELATED APPLICATIONS

The present application is a divisional of and claims priority to U.S. Ser. No. 19/092,012 filed Mar. 27^{th}, 2025, titled “SMART ADAPTIVE NEUROMODULATION DEVICE FOR STELLATE GANGLION MODULATION,” which is hereby incorporated by reference.

### TECHNICAL FIELD

The embodiments generally relate to the technical field of non-invasive neuromodulation of the stellate ganglion using real-time biometric feedback.

### BACKGROUND

The stellate ganglion plays a critical role in regulating the autonomic nervous system, particularly in conditions involving sympathetic overactivity such as chronic pain, post-traumatic stress disorder (PTSD), and autonomic dysregulation. Conventional treatments for stellate ganglion modulation, such as stellate ganglion block (SGB) and radiofrequency ablation (RFA), involve invasive procedures that carry risks of complications, including infection, nerve damage, and temporary or permanent loss of function.

Non-invasive neuromodulation techniques, such as transcutaneous electrical nerve stimulation (TENS) and transcutaneous vagus nerve stimulation (tVNS), have been explored but lack the specificity, depth of penetration, and adaptive control necessary for effective stellate ganglion modulation. Existing neuromodulation devices often operate with predefined stimulation parameters and lack real-time adaptability based on a patient's physiological response, limiting their efficacy and personalization.

Accordingly, there is a need for a non-invasive neuromodulation device that can precisely target the stellate ganglion while continuously adapting stimulation parameters in real-time based on biometric feedback. The present invention addresses this need by providing a wearable device capable of delivering at least one of low-intensity focused ultrasound (LIFU), radiofrequency (RF), or pulsed electromagnetic field (PEMF) energy to the stellate ganglion, while integrating real-time biometric monitoring and closed-loop feedback control to optimize therapy for individual patients.

### SUMMARY

This summary is provided to introduce a variety of concepts in a simplified form that is further disclosed in the detailed description of the embodiments. This summary is not intended to identify key or essential inventive concepts of the claimed subject matter, nor is it intended to determine the scope of the claimed subject matter.

The present invention provides a non-invasive neuromodulation device for modulating the stellate ganglion using real-time biometric feedback. The device includes a wearable collar configured to be positioned around a user's neck, a transducer array integrated into the collar, and a control unit with a microprocessor. The transducer array delivers at least one of low-intensity focused ultrasound, radiofrequency, or pulsed electromagnetic field energy to the stellate ganglion. Mapping of the location of the stellate ganglion occurs through ultrasound guidance.

The invention further includes biometric sensors configured to measure physiological parameters such as heart rate variability and skin conductance. A feedback system is operatively connected to the control unit, allowing the microprocessor to dynamically adjust neuromodulation parameters based on real-time biometric data. This closed-loop system optimizes stimulation intensity and duration to enhance therapeutic efficacy while preventing overstimulation.

The device may also include a wireless communication module that transmits data to an external interface, such as a mobile application or clinical monitoring system, allowing for remote monitoring and therapy adjustments. The system can be used for managing chronic pain, post-traumatic stress disorder, and autonomic dysregulation.

The device may also include a wired system for more complex energy delivery systems like LIFU.

In another aspect, the invention provides a method for non-invasively modulating the stellate ganglion. The method includes positioning a wearable device over the stellate ganglion region, delivering neuromodulatory stimulation using at least one of low-intensity focused ultrasound, radiofrequency, or pulsed electromagnetic field energy, and continuously monitoring physiological responses. The method further includes adjusting stimulation parameters in real-time using a closed-loop control system based on monitored biometric data to provide personalized therapy.

In yet another aspect, the invention provides a system for adaptive neuromodulation of the stellate ganglion, comprising a flexible, ergonomically designed wearable device, a multimodal neuromodulation unit, a sensor array, a machine learning-enabled control unit, and a wireless communication module. The system dynamically refines stimulation settings over time to improve long-term therapeutic outcomes.

Other illustrative variations within the scope of the invention will become apparent from the detailed description provided hereinafter. The detailed description and enumerated variations, while disclosing optional variations, are intended for purposes of illustration only and are not intended to limit the scope of the invention.

### BRIEF DESCRIPTION OF THE DRAWINGS

A more complete understanding of the embodiments, and the attendant advantages and features thereof, will be more readily understood by references to the following detailed description when considered in conjunction with the accompanying drawings wherein:

FIG. 1 illustrates a smart adaptive neuromodulation device for stellate ganglion modulation, according to some embodiments;

FIG. 2 illustrates a smart adaptive neuromodulation device for stellate ganglion modulation, according to some embodiments; and

FIG. 3 illustrates a smart adaptive neuromodulation device for stellate ganglion modulation, according to some embodiments.

### DETAILED DESCRIPTION

The specific details of the single embodiment or variety of embodiments described herein are set forth in this application. Any specific details of the embodiments described herein are used for demonstration purposes only, and no unnecessary limitation(s) or inference(s) are to be understood or imputed therefrom.

Before describing exemplary embodiments in detail, it is noted that the embodiments reside primarily in combinations of components related to devices and systems. Accordingly, the device components have been represented where appropriate by conventional symbols in the drawings, showing only those specific details that are pertinent to understanding the embodiments of the present disclosure so as not to obscure the disclosure with details that will be readily apparent to those of ordinary skill in the art having the benefit of the description herein.

A non-invasive neuromodulation device may modulate the stellate ganglion using real-time biometric feedback. The device may include a wearable collar, a transducer array, biometric sensors, a control unit, and a wireless communication module. The wearable collar may conform to the user's neck for positioning over the stellate ganglion, facilitating energy delivery. The collar may be formed from hypoallergenic, flexible materials, such as medical-grade silicone or polymer composites, to enhance comfort during extended use. An adjustable clasp mechanism may provide a secure fit and accommodate variations in neck size.

A transducer array may be integrated into the inner surface of the collar and positioned to direct neuromodulatory energy toward the stellate ganglion. The transducer array may include at least one low-intensity focused ultrasound, radiofrequency, or pulsed electromagnetic field energy emitter. The energy transmission range may be optimized to allow penetration through soft tissue to the stellate ganglion at depths of approximately 5 to 10 millimeters using ultrasound guidance for mapping of the stellate and directing the energy.

Low-intensity focused ultrasound transducers may operate within a frequency range of 0.5 to 3 megahertz, generating focused acoustic stimulation while mitigating excessive thermal effects. Radiofrequency emitters may generate energy within a frequency range of 300 kilohertz to 1 megahertz, allowing deeper tissue penetration. Pulsed electromagnetic field generators may operate within the 1 to 100 hertz range, producing low-frequency electromagnetic fields that may influence nerve activity. The transducer array may be arranged to provide spatially selective modulation, using multiple emitters positioned to support uniform and controlled energy delivery across the stellate ganglion.

Biometric sensors may be embedded within the collar, positioned for skin contact to continuously monitor physiological parameters associated with autonomic nervous system activity. A heart rate variability sensor may use photoplethysmography or electrocardiography electrodes to measure variations in the time interval between heartbeats. An electrodermal activity sensor may detect changes in skin conductance. A blood pressure sensor, when included, may assess sympathetic activity using pulse wave analysis or oscillometric methods. A respiration rate sensor may measure thoracic impedance changes to track breathing patterns.

A control unit may include a microprocessor configured to process real-time biometric data and adjust neuromodulation parameters. The control unit may receive physiological input from biometric sensors, process data using machine learning algorithms, and regulate stimulation intensity, frequency, and duration in response. The control unit may analyze trends in heart rate variability and electrodermal activity, modify transducer output parameters based on detected autonomic nervous system activity, adjust energy levels to mitigate overstimulation, and refine stimulation patterns over multiple sessions using a closed-loop feedback process.

A wireless or wired communication module may allow for real-time remote monitoring and therapy adjustments. The device may transmit biometric data and stimulation parameters to a connected Bluetooth or Wi-Fi-enabled interface, such as a mobile application or clinical dashboard. A remote interface may provide healthcare providers with access to therapy settings, real-time physiological responses, and stored session data.

A rechargeable lithium-ion battery may supply power for up to four to six hours per charge. A USB-C or wireless charging dock may provide recharging functionality. A user interface may include physical control buttons for starting, pausing, or stopping treatment, along with LED indicators that may convey operational status, battery level, or stimulation intensity.

A user may position the wearable collar around the neck, ensuring alignment with the stellate ganglion. Upon activation, the device may initiate a calibration phase, allowing biometric sensors to establish baseline physiological readings. Once calibration is complete, the transducer array may deliver neuromodulatory stimulation according to predefined therapy parameters. Ultrasound guidance may be used to appropriately map and calibrate delivery of the neuromodulatory stimulation.

Throughout a session, biometric sensors may continuously monitor physiological responses, relaying data to the control unit for real-time analysis. The control unit may adjust stimulation parameters based on detected physiological changes, allowing autonomic regulation to adapt dynamically.

A method for non-invasive neuromodulation of the stellate ganglion may include positioning a wearable device over the stellate ganglion region, delivering neuromodulatory stimulation using at least one of low-intensity focused ultrasound, radiofrequency, or pulsed electromagnetic field energy, and continuously monitoring physiological responses. The method may further include adjusting stimulation parameters in real time using a closed-loop control process.

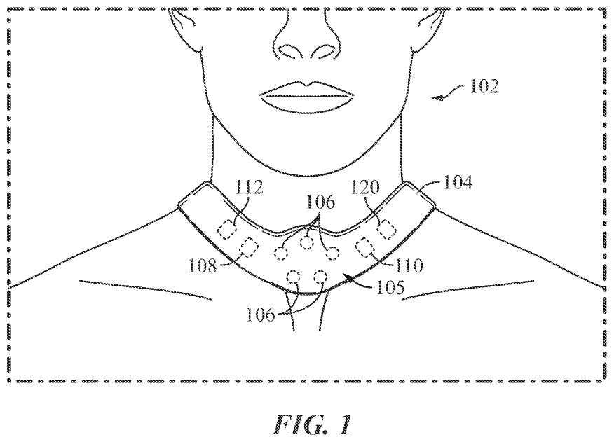

FIG. 1 illustrates a perspective view of a non-invasive neuromodulation device worn by user 102. Wearable collar 104 is positioned around the user's neck and is designed to direct neuromodulatory stimulation toward stellate ganglion region 105. Integrated within the wearable collar 104 is transducer array 106, which includes at least one low-intensity focused ultrasound emitter. One or more biometric sensors 108 are positioned within the collar for continuous monitoring of physiological parameters. Control unit 110 is operatively connected to the biometric sensors 108 and a feedback system 112, which dynamically adjusts the stimulation parameters of transducer array 106 based on biometric data detected by the biometric sensors 108.

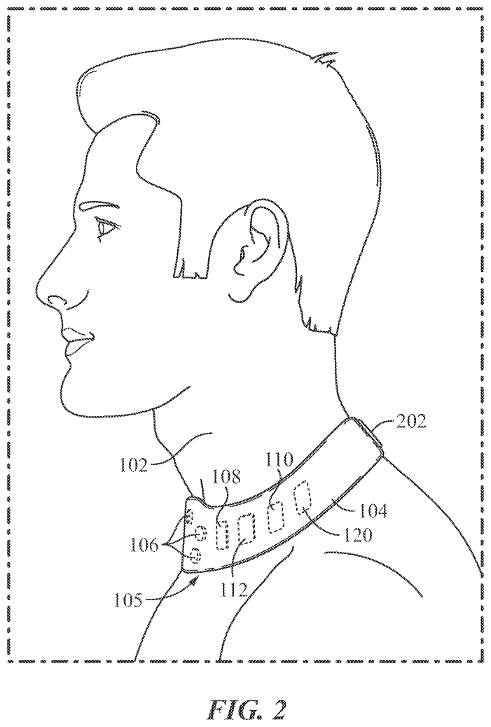

FIG. 2 illustrates another view of the neuromodulation device, highlighting a fastening mechanism 202 that allows for putting on or taking off the wearable collar 104 from a user's 102 neck. The wearable collar 104 houses transducer array 106 and one or more biometric sensors 108, as well as control unit 110 configured to process real-time biometric feedback from biometric sensors 108 and adjust stimulation settings accordingly. Feedback system 112 enables closed-loop modulation to optimize therapy effectiveness. One or more batteries 120 may power any of the transducer array 106, biometric sensors 108, control unit 110, feedback system 112, etc.

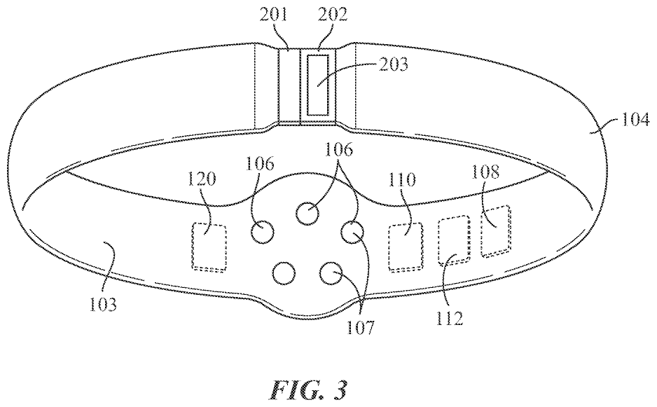

FIG. 3 illustrates a detailed view of the wearable collar 104 and its structural components. Clip 201 and buckle 203 provide an adjustable fastening mechanism 202, allowing the collar to conform to different neck sizes while ensuring consistent positioning over stellate ganglion region 105. Fastening mechanism 202 secures the collar's structural integrity. Control unit 110 is integrated into the collar and connected to transducer array 106, which houses at least one low-intensity focused ultrasound emitter 107. One or more biometric sensors 108 are embedded within the collar for continuous physiological monitoring. Feedback system 112 processes real-time data and adjusts stimulation parameters accordingly.

In this disclosure, the descriptions of the various embodiments have been presented for purposes of illustration and are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein. Thus, the appended claims should be construed broadly, to include other variants and embodiments, which may be made by those skilled in the art.

It will be appreciated by persons skilled in the art that the present embodiment is not limited to what has been particularly shown and described hereinabove. A variety of modifications and variations are possible considering the above teachings without departing from the following claims.

What is claimed is:

1. A method for non-invasively modulating a stellate ganglion, the method comprising:
positioning a wearable device with an integrated transducer array over a stellate ganglion region of a user;
delivering neuromodulatory stimulation using at least one of a low-intensity focused ultrasound (LIFU), radiofrequency, or pulsed electromagnetic field energy;
continuously monitoring physiological responses, including heart rate variability and skin conductance, via embedded sensors;
adjusting stimulation parameters in real-time using a closed-loop control system based on monitored physiological data; and
providing adaptive neuromodulation personalized to a user's autonomic response.

2. The method of claim 1, further comprising the step of logging biometric data over multiple therapy sessions to generate a personalized neuromodulation profile.

3. The method of claim 1, wherein the wearable device provides haptic or auditory feedback to indicate changes in stimulation intensity.

4. The method of claim 1, wherein the wearable device is used as a preventive treatment for autonomic dysfunction in individuals predisposed to conditions such as PTSD or chronic pain.

5. The method of claim 1, wherein the system integrates with telehealth platforms to allow remote physician oversight.

6. A system for adaptive neuromodulation of a stellate ganglion, comprising:
a flexible, ergonomically designed wearable device configured to be positioned on a user's neck;
a multimodal neuromodulation unit comprising LIFU, radiofrequency, or pulsed electromagnetic field emitters;
a sensor array including at least a heart rate variability sensor and an electrodermal activity sensor for measuring autonomic nervous system activity;
a machine learning-enabled control unit configured to process real-time sensor data and dynamically modulate neuromodulation parameters; and
a wireless communication module configured to transmit data to an external interface for remote monitoring and therapy adjustments.

7. The system of claim 5, wherein the control unit wirelessly communicates with a mobile application that provides real-time visualization of autonomic nervous system responses.

8. The system of claim 5, wherein the neuromodulation unit includes a mode for delivering alternating stimulation patterns to prevent neural habituation.

9. The system of claim 5, wherein the neuromodulation unit operates at an energy level optimized for safety and long-term therapeutic use.

10. The system of claim 5, wherein the machine learning-enabled control unit refines stimulation settings over time to improve long-term therapeutic outcomes.
